(19) United States
(12) Patent Application Publication
(10) Pub. No.: US 2011/0146681 A1
(43) Pub. Date: Jun. 23, 2011

(54) Adaptive Flow Sensor Model

(75) Inventors: Mehdi M. Jafari, Laguna Hills, CA (US); Rhomere S. Jimenez, San Diego, CA (US); Edward McCoy, Vista, CA (US); Gail F. Upham, Fallbrook, CA (US)

(73) Assignee: Nellcor Puritan Bennett LLC, Boulder, CO (US)

(21) Appl. No.: 12/643,083

(22) Filed: Dec. 21, 2009

(51) Int. Cl. A61M 16/00

(52) U.S. Cl. 128/204.21

(57) ABSTRACT

The disclosure describes a novel approach of estimating patient sensor data for sensors in sensor tubing or sensor lines during purging or autozeroing or any other situations under which no measurement is provided by the sensor.

### INTRODUCTION

Medical ventilators may determine when a patient takes a breath in order to synchronize the operation of the ventilator with the natural breathing of the patient. In some instances, detection of the onset of inhalation and/or exhalation may be used to trigger one or more actions on the part of the ventilator.

In order to accurately detect the onset of inhalation and/or exhalation, and/or obtain a more accurate measurement of inspiratory and expiratory flow/volume, a flow or pressure sensor may be located close to the patient. For example, to achieve accurate and timely non-invasive signal measurements, differential-pressure flow transducers may be placed at the patient wye proximal to the patient. However, the ventilator circuit and particularly the patient wye is a challenging environment to make continuously accurate measurements. The harsh environment for the sensor is caused, at least in part, by the condensations resulting from the passage of humidified gas through the system as well as secretions emanating from the patient. Over time, the condensate material can enter the sensor tubes and/or block its ports and subsequently jeopardize the functioning of the sensor.

One approach to address this issue involves sending a puff, pocket, or purge of air down the differential pressure sensing lines away from the ventilator. Such a purge may help remove unwanted condensate or the like from the lines and/or from the proximal flow sensor package. In addition, periodic calibrations (autozero) are needed to update sensor parameters (e.g. pressure transducers zero basis). During episodes of purging or autozeroing, the sensor does not provide any pressure or flow measurement readings. The purging and autozeroing events are often scheduled frequently. Accordingly, during ventilation, there are frequent and relatively long intervals (especially in the case of neonatal patients) of missing proximal flow and pressure data.

### SUMMARY

The disclosure describes a novel approach of estimating patient sensor data for sensors in sensor tubing or sensor lines during purging or autozeroing or any other situations under which no measurement is provided by the sensor.

Based on this approach, an adaptive internal model of the proximal sensor readings (flow and pressure measurements) is developed using the internally available measurements, settings, and hardware characteristic parameters. This model is intended to simulate the actual sensor (physically located at the patient wye). The model parameters are adaptively adjusted to match the actual sensor readings. During normal operation, model parameters are optimized to minimize the deviation between the actual and simulated performance. In the absence of readings from the physical sensor, the updated sensor model may be used instead to obtain simulated readings for operational use.

In part, this disclosure describes a method for estimating at least one patient reading from at least one circuit sensor in a medical ventilator providing ventilation to a patient. The method includes performing the following steps:

a) monitoring a plurality of sensors including a first sensor and at least one second sensor to obtain first sensor measurements and second sensor measurements;

b) fitting a curve for first sensor measurements versus time based on the second sensor measurements, wherein said fitting relies on one or more fit parameters, and wherein the values of said one or more fit parameters are found by said fitting;

e) calculating an estimate of a first sensor measurements for a time interval based on the one or more fit parameters and the second sensor measurements; and

d) displaying the estimate of the first sensor measurements for the time interval instead of the first sensor measurements.

Yet another aspect of this disclosure describes a pressure support system that includes: a processor; a pressure generating system adapted to generate a flow of breathing gas controlled by the processor; a ventilation system including a patient circuit is controlled by the processor; at least one circuit sensor in fluid communication with the patient circuit via one or more sensor tubes; and an adaptive flow sensor model is controlled by the processor, the adaptive flow sensor model is adapted to estimate patient sensor data during situations in which the at least one circuit sensor cannot obtain a reading.

In yet another aspect, the disclosure describes a medical ventilator system that includes: a processor; a gas regulator is controlled by the processor, the gas regulator adapted to regulate a flow of gas from a gas supply to a patient via a patient circuit; a flow sensor package disposed in the patient circuit, the flow sensor package is controlled by a gas accumulator; a pressure sensor coupled to the gas accumulator and is controlled by the processor, the pressure sensor adapted to provide pressure readings in the gas accumulator to the processor; and an adaptive flow sensor model is controlled by the processor for estimating patient sensor data when the flow sensor and pressure sensor are not reading patient data.

These and various other features as well as advantages which characterize the systems and methods described herein will be apparent from a reading of the following detailed description and a review of the associated drawings. Additional features are set forth in the description which follows, and in part will be apparent from the description, or may be learned by practice of the technology. The benefits and features of the technology will be realized and attained by the structure particularly pointed out in the written description and claims hereof as well as the appended drawings.

It is to be understood that both the foregoing general description and the following detailed description are exemplary and explanatory and are intended to provide further explanation of the invention as claimed.

### BRIEF DESCRIPTION OF THE DRAWINGS

The following drawing figures, which form a part of this application, are illustrative of embodiments systems and methods described below and are not meant to limit the scope of the invention in any manner, which scope shall be based on the claims appended hereto.

FIG. 1 illustrates an embodiment of a ventilator connected to a human patient.

FIG. 2 illustrates an embodiment of a proximal sensor module that includes a sensor purging system and an adaptive proximal flow sensor model.

FIG. 3 illustrates an embodiment of a method for at least one patient reading from at least one circuit sensor in a medical ventilator providing ventilation to a patient.

FIG. 4 illustrates an embodiment of a method for at least one patient reading from at least one circuit sensor in a medical ventilator providing ventilation to a patient.

### DETAILED DESCRIPTION

Although the techniques introduced above and discussed in detail below may be implemented for a variety of medical devices, the present disclosure will discuss the implementation of these techniques in the context of a medical ventilator for use in providing ventilation support to a human patient. The reader will understand that the technology described in the context of a medical ventilator for human patients could be adapted for use with other systems such as ventilators for non-human patients and general gas transport systems in which sensor tubes in challenging environments may require periodic or occasional purging, autozeroing or other types of operations that would render their primary measurement function inoperative.

Medical ventilators are used to provide a breathing gas to a patient who may otherwise be unable to breathe sufficiently. In modern medical facilities, pressurized air and oxygen sources are often available from wall outlets. Accordingly, ventilators may provide pressure regulating valves (or regulators) connected to centralized sources of pressurized air and pressurized oxygen. The regulating valves function to regulate flow so that respiratory gas having a desired concentration of oxygen is supplied to the patient at desired pressures and rates. Ventilators capable of operating independently of external sources of pressurized air are also available.

While operating a ventilator, it is desirable to monitor the rate at which breathing gas is supplied to the patient. Accordingly, systems typically have interposed flow and/or pressure sensors. The sensors may be connected to or is controlled by the inspiratory limb and the expiratory limb of the ventilator and/or patient circuit. In some cases, it is desirable to provide a flow sensor and/or pressure sensor near the wye of the patient circuit, which connects the inspiratory limb and the expiratory limb near the patient interface (e.g., an endotracheal tube, mask, or the like). Such a sensor package may be referred to as a proximal sensor system, device or module.

During operation, the patient circuit can acquire exhaled condensate from the patient and/or condensate from the action of a humidifier in the patient circuit. For circuits containing a proximal flow sensor package which measures flow using the principle of differential pressure, the presence of such liquid or viscous material in either or both of the lines used to sense differential pressure can reduce sensor performance. One approach to address this issue involves sending a puff, pocket, or discharge of air down each of the differential pressure sensing tubes. Such a discharge, which may also be referred to as a single or individual purge of the tube, may help remove or prevent unwanted condensate or the like from the tubes and/or from the proximal flow sensor package. Depending on the embodiment, purging is performed using a sensor tube purge system or module which may be integral with the proximal sensor module or a separate and independent system.

The proximal flow sensor or pressure sensor may be disconnected, disabled, or connected to a pressurized vessel during purging to prevent the pressure sensor from being damaged by the abrupt change in pressure and from reading and recording the change in pressure caused by sending the puff, pocket, or discharge of air down each of the differential pressure sensing tubes. In addition, periodic calibrations (autozero) are needed to update sensor parameters (e.g. pressure transducers zero basis). During episodes of purging or autozeroing, the sensor does not provide any pressure or flow measurement readings. Out of necessity the purging and autozeroing events are scheduled frequently causing frequent and relatively long intervals (especially in the case of neonatal patients) of missing proximal flow and pressure data. To prevent this gap in data a proximal flow sensor model may be utilized to simulate ongoing ventilator measurements and settings, such as proximal wye pressure and flow. The model parameters are based on ventilator setting and hardware characteristics. The values for the model parameters are adaptively adjusted based on the actual proximal sensor readings during normal operation to minimize the difference between simulated estimates and actual readings.

When a proximal sensor monitoring device is integrated with a ventilator, it is desirable to add functionality to coordinate the operation of the system as an integrated whole. In addition, functionality can be added to provide more information to ensure satisfactory operation of proximal sensor monitoring at all times. The addition of such improvements can result in an integrated system well-tuned to the features of the ventilator, with higher reliability, improved performance, and consequently, improved patient outcomes.

Those skilled in the art will recognize that the methods and systems of the present disclosure may be implemented in many manners and as such are not to be limited by the foregoing exemplary embodiments and examples. In other words, functional elements being performed by a single or multiple components, in various combinations of hardware and software or firmware, and individual functions, can be distributed among software applications at either the client or server level or both. In this regard, any number of the features of the different embodiments described herein may be combined into single or multiple embodiments, and alternate embodiments having fewer than or more than all of the features herein described are possible. Functionality may also be, in whole or in part, distributed among multiple components, in manners now known or to become known. Thus, myriad software/hardware/firmware combinations are possible in achieving the functions, features, interfaces and preferences described herein. Moreover, the scope of the present disclosure covers conventionally known manners for carrying out the described features and functions and interfaces, and those variations and modifications that may be made to the hardware or software or firmware components described herein as would be understood by those skilled in the art now and hereafter.

As discussed above, during purging a sensor is removed from the purge flow, disabled, or disconnected. Accordingly, the sensor does not read or send any patient data during purging causing frequent patient data gaps. The adaptive proximal flow sensor model estimates what the patient data or sensor readings would be during these purges. These estimates are saved, sent, and/or displayed by the ventilator eliminating gaps in patient sensor data caused by purging or other measurement disruptive events. These readings provide care-givers, patients, and the ventilators with more comprehensive and continuously available information and allow for more informed patient treatment and diagnoses. In an embodiment, the proximal flow and pressure at patient circuit wye are estimated by utilizing first sensor measurements versus time based on second sensor measurements in a fitting curve. The fitting relies on one or more fit parameters and the one or more fit parameters are found by fitting. In one embodiment, the proximal flow and pressure at patient circuit wye are estimated by utilizing the following model equations:

P_{y}(t)=Pₑₓₕ(t)+Q_{c}(t)*(K₁+K₂*Q_{c}(t)); and

Q_{c}(t)=Qₑₓₕ(t)+C_{ef}*\dot{P}ₑ(t).

Wherein:

P_{y}=pressure at patient circuit wye, which is measured by the proximal sensor;

Q_{c}=flow rate in the exhalation limb, which is derived or calculated utilizing the above equation;

C_{ef}=compliance of exhalation filter and is a determined constant;

K₁, K₂=parameters of exhalation circuit limb resistance and are modeling parameters for the flow going through the circuit;

Pₑₓₕ=pressure reading at the exhalation port, which is measured by an exhalation port sensor;

Qₑₓₕ=flow reading at exhalation port, which may be directly measured or determined from a differential pressure sensor;

t=a continuous variable and stands for time in seconds as it elapses;

P_{y}(t)=the wye pressure estimate at time t; and

\dot{P}ₑ=conditioned (filtered) time domain derivative of pressure (rate of change of pressure with time) measured at exhalation port, this slope may be calculated utilizing the following model equations in the frequency domain:

$${\dot{P}}_{e} ( s ) = \frac{s}{( s + {p}_{1} ) ( s + {p}_{2} ) ( β s + 1 )} {P}_{e} ( s ) ;$$

$${Q}_{y} ( s ) = {T}_{1} ( s ) * {Q}_{v} ( s ) + {T}_{2} ( s ) * {P}_{y} ( s ) + {E}_{Qy} ( s ) ;$$

Pₑ=measured pressure at the exhalation port

Q_{y}(t)=estimated proximal flow at the patient circuit wye;

Qᵥ(t)=Qdel(t)−Qₑₓₕ(t);

Q_{del}(t)=total flow delivered by the ventilator;

E_{Qy}(t)=approximation residual or estimation error;

Q_{y}(s)=Laplace transform of the flow rate at the patient circuit wye;

T₁(s)Qᵥ(s)=the Laplace transform of the contribution of the ventilator flow rate to the patient flow rate;

T₂(s)*P_{y}(s)=the Laplace transform of the contribution of pressure at patient circuit wye to patient flow rate;

$${T}_{1} ( s ) = α \frac{ s + {z}_{1}}{( s + {p}_{3} ) ( s + {p}_{4} )} ; and$$

$${T}_{2} ( s ) = - m * {T}_{1} ( s ) * \frac{s}{( s + {p}_{5} ) ( s + {p}_{6} )} .$$

Model parameters α, β, c, z, p, and m are dynamically updated to optimize the match between actual and simulated readings over a regular cycle, such as one breath. Additionally, one or more of these parameters may assume different values depending on the breath phase (inhalation or exhalation). The values for the model parameters are adaptively adjusted based on the actual proximal sensor readings and readings from the other sensors in the system during normal operation to minimize the difference between the simulated estimates and actual proximal sensor readings.

The model described above is but one example of how an estimate may be obtained during periods in which the proximal sensor output is not available based the prior readings of the proximal sensor and the prior and current readings of other sensors in the ventilator. Alternative model parameters and more involved optimization strategies may also be used. Furthermore, other wave-shaping modeling approaches and waveform quantifications and modeling techniques may be utilized. Moreover, parameters of such models may be dynamically updated and optimized during normal sensor operation to obtain the least difference between actual and simulated signals.

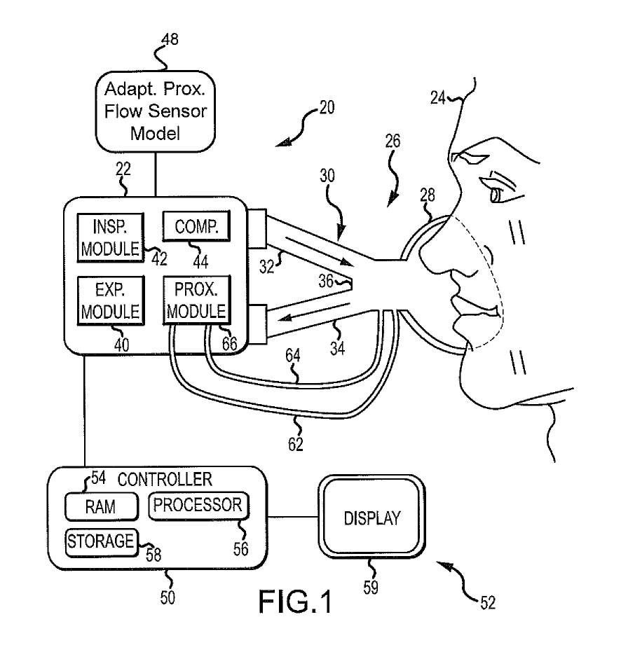

FIG. 1 illustrates an embodiment of a ventilator 20 connected to a human patient 24. Ventilator 20 includes a pneumatic system 22 (also referred to as a pressure generating system 22) for circulating breathing gases to and from patient 24 via the ventilation tubing system 26, which couples the patient 24 to the pneumatic system 22 via physical patient interface 28 and ventilator circuit 30. Ventilator circuit 30 could be a two-limb or one-limb circuit for carrying gas to and from the patient. In a two-limb embodiment as shown, a wye fitting 36 may be provided as shown to couple the patient interface 28 to the inspiratory limb 32 and the expiratory limb 34 of the circuit 30.

The present systems and methods have proved particularly advantageous in invasive settings, such as with endotracheal tubes. However, condensation and mucus buildup do occur in a variety of settings, and the present description contemplates that the patient interface may be invasive or non-invasive, and of any configuration suitable for communicating a flow of breathing gas from the patient circuit to an airway of the patient. Examples of suitable patient interface devices include a nasal mask, nasalloral mask (which is shown in FIG. 1), nasal prong, full-face mask, tracheal tube, endotracheal tube, nasal pillow, etc.

Pneumatic system 22 may be configured in a variety of ways. In the present example, system 22 includes an expiratory module 40 coupled with an expiratory limb 34 and an inspiratory module 42 coupled with an inspiratory limb 32. Compressor 44 or another source or sources of pressurized gas (e.g., pressured air and/or oxygen controlled through the use of one or more gas regulators) is coupled with inspiratory module 42 to provide a source of pressurized breathing gas for ventilatory support via inspiratory limb 32.

The pneumatic system may include a variety of other components, including sources for pressurized air and/or oxygen, mixing modules, valves, sensors, tubing, accumulators, filters, etc. Controller 50 is operatively coupled with pneumatic system 22, signal measurement and acquisition systems, and an operator interface 52 may be provided to enable an operator to interact with the ventilator (e.g., change ventilator settings, select operational modes, view monitored parameters, etc.). Controller 50 may include memory 54, one or more processors 56, storage 58, and/or other components of the type commonly found in command and control computing devices.

The memory 54 is computer-readable storage media that stores software that is executed by the processor 56 and which controls the operation of the ventilator 20. In an embodiment, the memory 54 comprises one or more solid-state storage devices such as flash memory chips. In an alternative embodiment, the memory 54 may be mass storage connected to the processor 56 through a mass storage controller (not shown) and a communications bus (not shown). Although the description of computer-readable media contained herein refers to a solid-state storage, it should be appreciated by those skilled in the art that computer-readable storage media can be any available media that can be accessed by the processor 56. Computer-readable storage media includes volatile and non-volatile, removable and non-removable media implemented in any method or technology for storage of information such as computer-readable instructions, data structures, program modules or other data. Computer-readable storage media includes, but is not limited to, RAM, ROM, EPROM, EEPROM, flash memory or other solid state memory technology, CD-ROM, DVD, or other optical storage, magnetic cassettes, magnetic tape, magnetic disk storage or other magnetic storage devices, or any other medium which can be used to store the desired information and which can be accessed by the processor 56.

As described in more detail below, controller 50 issues commands to pneumatic system 22 in order to control the breathing assistance provided to the patient by the ventilator. The specific commands may be based on inputs received from patient 24, pneumatic system 22 and sensors, operator interface 52 and/or other components of the ventilator. In the depicted example, operator interface includes a display 59 that is touch-sensitive, enabling the display to serve both as an input user interface and an output device.

The ventilator 20 is also illustrated as having a proximal sensor module (the “Prox. Module” in FIG. 1) 66. The proximal sensor module 66 includes at least one sensor, such as a pressure sensor, that is connected to some location in the patient circuit 30 or patient interface 28 by one or more sensor tubes 62, 64. In the embodiment shown, two sensor tubes 62, 64 connect the proximal sensor module 66 to a location in the wye fitting 36. As is known in the art, for the differential pressure measurement system to operate, a resistance to flow is placed between the flow outlets of the two sensor tubes 62, 64. In alternative embodiments, sensor tubes may connect to the ventilator tubing system 26 at any location including any limb of the circuit 30 and the patient interface 28. It should be noted that regardless of where the sensor tubes connect to the tubing system 26, because it is assumed that there is very little or no leakage from the tubing system 26 all gas discharged through the sensor tubes into the ventilator tubing system 26 will ultimately be discharged from the ventilator through the patient circuit 30 and expiratory module 40. The use of sensor tubes as part of various different measurement systems is known in the art.

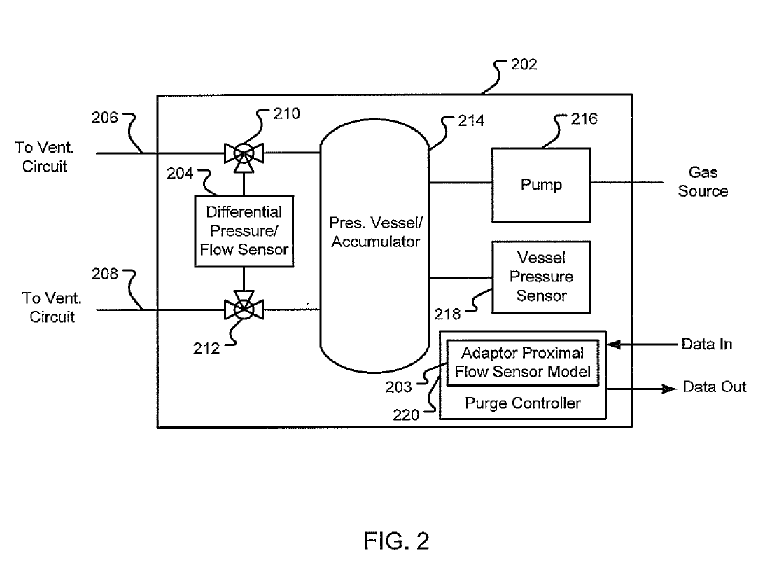

In the embodiment shown, the proximal sensor module 66 includes a sensor tube purging system that purges the sensor tubes by repeatedly discharging gas through the sensor tubes into the ventilator circuit 30. The sensor tube purging system and functions are discussed in greater detail with reference to FIG. 2.

Further, in the embodiment shown, the controller 55 utilizes the ongoing measurements taken by the proximal sensor module 66 and the ventilator settings in an adaptive proximal flow sensor model 48 to simulate patient sensor readings during purging or other measurement disruptive events. The adaptive proximal flow sensor model 48 may be based on inputs received from patient 24, pneumatic system 22 and sensors, operator interface 52 and/or other components of the ventilator. The adaptive proximal flow sensor model 48 can be stored in and utilized by the controller 55, by a computer system located in the proximal sensor module 66, or by an independent source that is operatively coupled with the pneumatic system 22 as shown in FIG. 1. The adaptive proximal flow sensor model 48 may also interact with the signal measurement and acquisition systems, the controller 50 and the operator interface 52 to enable an operator to interact with the module, the model, the ventilator, and the display. Further, this coupling allows the controller to receive and display the estimated patient sensor readings produced by the adaptive proximal flow sensor model 48. This computer system may include memory, one or more processors, storage, and/or other components of the type commonly found in command and control computing devices.

Although FIG. 1 illustrates an embodiment having two sensor tubes 62, 64 and one proximal sensor module 66, any number of sensor tubes may be used depending on the number and types of proximal sensors. For example, in some embodiments module 66 couples to three (3) tubes, with two (2) tubes used for a differential pressure sensor function and the third tube used for an alternative function such as gas composition analysis, orientation or other alternative sensors, or the like. All of the sensors may be housed in a single proximal sensor module 66 or they may be separated into different modules 66.

Furthermore, a proximal sensor module 66 may be integrated into the ventilator 20 as shown, or may be a completely independent module. If independent, the proximal flow module 66 may be adapted to detect the current phase of a patient's breathing cycle in order to synchronize the purging of the sensor tubes with specific breathing phases, such as the inspiratory phase or the exhalation phase or other conditions such as respiratory maneuvers or user-initiated purging.

Each proximal sensor module 66 may provide its own purging or a single sensor tube purge system may be provided, which may be a module incorporated into a proximal sensor module 66 or may be an independent purge module (e.g., a user-generated “purge now” command).

FIG. 2 illustrates an embodiment of a proximal sensor module 202 that includes a sensor tube purging system and an adaptive proximal flow sensor model 203. The proximal sensor module 202 and/or the adaptive proximal flow sensor model 203 may be implemented as an independent, stand-alone module, e.g., as a separate card either inside the ventilator or within a separate housing associated with the proximal flow sensor. Alternatively, the proximal sensor module 202 and/or the adaptive proximal flow sensor model 203 may be integrated with components of the ventilator or another device, e.g., built into a ventilator control board. In yet another embodiment, the sensor tube purge system may be implemented independently from the proximal sensor 204, for example as an in-line module between the sensor and the patient circuit, in which case the module of FIG. 2 would not include the proximal sensor 204.

In the embodiment shown, a proximal sensor module 202 is illustrated having a differential pressure/flow sensor 204 connected to two sensor tubes 206, 208 that are subsequently attached to the ventilator tubing system (not shown). Sensor tubes used in conjunction with proximal sensors may have relatively small internal diameters. For example, tube diameters may be less than about 10 millimeters (mm), less than about 1 mm, or even smaller. Such sensor tubes are prone to blockage and, also because of their small diameters, are relatively more detrimentally affected by inner surface contamination even when not completely occluded.

In the embodiment shown, the differential pressure sensor 204 is connected to each sensor tube 206, 208 by a corresponding valve 210, 212. The valves 210, 212 are also connected to a pressurized vessel 214, sometimes also referred to as an accumulator 214, and operate such that when a sensor tube 206, 208 is connected to the vessel 214 (thus allowing pressurized gas from the vessel to be discharged through the sensor tube to the ventilator circuit) the associated sensor tube is not connected to the pressure sensor 204. This protects the sensor 204 from damage due to the abrupt change in pressure caused when the sensor tube is purged. In another embodiment, when performing an individual purge of either sensor tube of a differential pressure sensor, the sensor is also disconnected from the both sensor tubes. In yet another embodiment, the differential pressure sensor is always connected to the sensor tubing regardless of whether the tubes are being purged or not. In this embodiment, the sensor 204 may or may not be disabled (turned off) to prevent damage or the recording of spurious pressure measurements.

As discussed above, when sensor 204 is removed from the purge flow, disabled, or disconnected, the sensor does not send or read any patient data causing frequent patient sensor data gaps. The adaptive proximal flow sensor model 203 estimates what the patient data or sensor readings would be during these purges. These estimates are saved, sent, and displayed in the ventilator eliminating gaps in patient sensor data. These readings provide care-givers, patients, and the ventilators with more comprehensive information and allow for more informed patient treatment and diagnoses.

In the embodiment shown, the purge module in the proximal sensor module 202 includes the accumulator 214, a pump 216 (or alternatively a source of pressurized gas and a regulator) for charging the accumulator 214 with gas obtained from an external source (e.g., ambient), a pressure sensor 218 for monitoring the pressure in the accumulator 214, the aforementioned valves 210, 212 and a purge controller 220 that controls the functions of the purge module. In this embodiment, the purge controller 220 includes the adaptive proximal flow sensor model 203. The accumulator 214 may be any appropriate size and rated to any appropriate pressure. In an embodiment, because the volumes and pressures necessary to purge the typically small-diameter sensor tubes are relatively small and cost and size are always important design factors, the accumulator 214 may have a volume between about five (5) milliliters (ml) to about 20 milliliters. In a specific embodiment, the accumulator 214 volume is between about 10 ml and about 12 ml. In some embodiments, accumulator 214 is rated to hold and/or maintain pressures between about two (2) pounds per square inch (PSI) and about thirty (30) pounds per square inch, with ratings of up to about 3 psi, up to about 6 psi and up to about 8 psi used in various embodiments depending on pump size. The pump 216 may be of any type and may receive filtered air or any other gas, including respiratory gas obtained directly from the ventilator.

For example, in an embodiment, when power is applied to the pump 216, gas from the gas source is pumped under pressure into the accumulator 214. When power is removed from the pump 216, the pump contains a suitable structure such that the pressure built up in the accumulator 214 does not discharge back through the pump. Such structure provides the function of a check valve without requiring an extra component.

In the embodiment shown, the accumulator pressure sensor 218 is provided to obtain information concerning the pressure within the vessel 214. From this information, the amount of gas used during purging can be determined. Depending on the embodiment, the raw pressure data may be provided to the ventilator for use in calculating the gas flow through the patient circuit or may be provided to the purge controller 220, which calculates the purge volume and provides that data to the ventilator. Such a calculation would be performed based on the pressure changes observed during the purge cycle and previously determined data characterizing the volume, compliance and other parameters of the purge module as is known in the art.

In the embodiment shown, the purge controller 220 controls the purging of the sensor tubes 206, 208 by controlling the opening and closing of the valves 210, 212 and the pressurizing of the accumulator 214 by the pump 216. Additionally, in the embodiment shown, the purge controller 220 further controls the utilization of the adaptive proximal flow sensor model 203. However, the purging and the adaptive proximal flow sensor model 203 may be controlled by any suitable component, such as the ventilator controller, a microprocessor, and a valve controller. In this embodiment, the purge controller 220 includes a microprocessor executing software stored either on memory within the processor or in a separate memory cache. The purge controller 220 transmits sensor data from the differential pressure/flow sensor 204 and sensor estimates from the adaptive proximal flow sensor model 203 to other devices or components such as the ventilator.

As discussed above, the controller 220 may also interface between the ventilator and the purge system to provide information such as the status of the purge system (e.g., currently discharging, time since last discharge, currently in a purge cycle, time since last purge cycle, purge failure error due to possible occlusion of a sensor tube, time/duration of last discharge, time until next discharge, current interval setting, component failure, etc.) and the amount of purge gas delivered into the patient circuit. The controller 220 may utilize this information in estimating the patient sensor data during purges. Further, the interface between the ventilator and the purge system can provide the ventilator with the simulated sensor estimates and provide the purge system with ventilator settings and sensor data for estimating patient sensor data during purging. Further, the controller 220 may update this information continuously in order to obtain accurate sensor estimates. The controller 220 may also receive information from external sources such as modules of the ventilator, in particular information concerning the current breathing phase of the patient, ventilator parameters and other ventilator readings. The information received may include user-selected or predetermined values for various parameters such as the purge cycle interval (e.g., perform a purge cycle every 10 minutes), accumulator pressure, between-discharges delay period, individual purge/discharge interval, sensor estimate interval, etc. The information received may further include directions such as a ventilator-generated purge command or sensor estimate command or an operator command to perform a purge cycle and sensor estimate at the next opportunity (e.g., an automatic or a manual purge command). The controller 220 may also include an internal timer so that individual purges and purge cycles and patient sensor data estimates for these purges can be performed at a user or manufacturer specified interval.

In another embodiment, the controller for a medical ventilator comprises a microprocessor, an adaptive flow sensor model designed to estimate patient sensor data, and a sensor tube purge module adapted to initiate a purge cycle that purges sensor tubes connected to sensors in the medical ventilator. In this embodiment, the purge cycle includes repeatedly discharging gas through the sensor tubes into a patient circuit and discontinuing any readings of the sensors. In one embodiment, each gas discharge has a fixed duration of less than 100 milliseconds and each gas discharge is separated from the prior gas discharge by not more than 300 milliseconds. It will be understood by one of skill in the art that this time frame can be modified based on the specific patient, ventilator parameters, and applications.

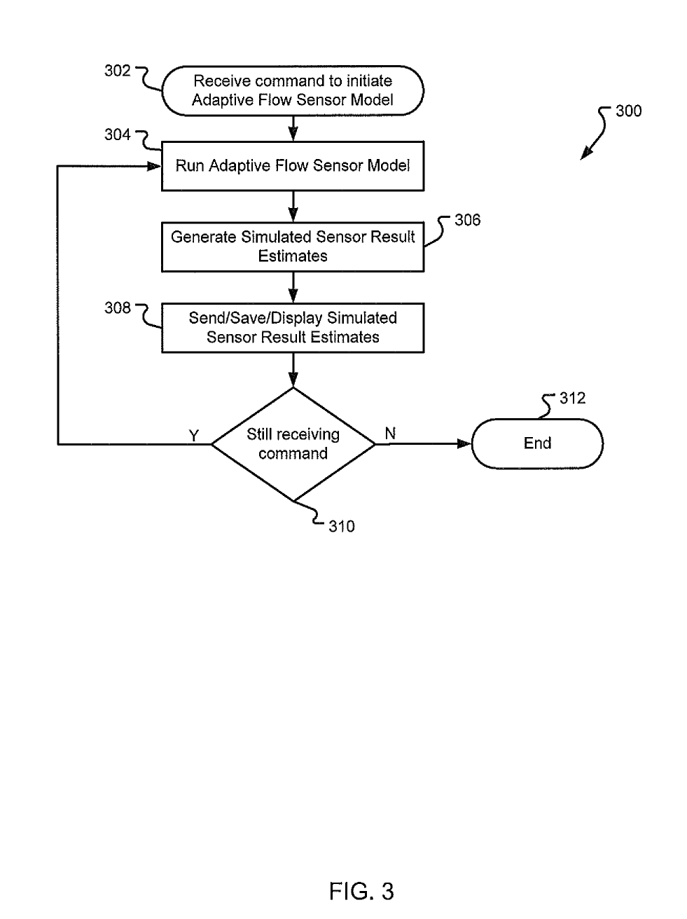

FIG. 3 represents and embodiment of a method for at least one patient reading from at least one circuit sensor in a medical ventilator providing ventilation to a patient, 300.

As illustrated, method 300 receives a command to initiate an adaptive flow sensor model, 302. In one embodiment, the command is from a controller, such as a pressure support system controller or a ventilator controller. In an alternative embodiment, the command is inputted by a user through a user interface. In another embodiment, the command is configured into the ventilator.

In response to this command, method 300 runs the adaptive flow sensor model, 304 and generates simulated sensor result estimates, 306. In one embodiment, the model utilizes past proximal flow and/or past pressure sensor measurements to generate simulated sensor result estimates. In a further embodiment, the model utilizes current and/or past ventilator sensor measurements and information to generate the simulated sensor result estimates. The model for the curve may be any suitable model as long as it can provide a reasonably accurate prediction of the pressure and/or flow at the wye based on past proximal sensor reading and current and/or past sensor readings for other sensors. In one embodiment, the model equations (in time and frequency domains) for the modeling process are:

$${P}_{y} ( t ) = {P}_{exh} ( t ) + {Q}_{c} ( t ) * ( {K}_{1} + {K}_{2} * {Q}_{c} ( t ) ) ;$$

$${Q}_{c} ( t ) = {Q}_{exh} ( t ) + {C}_{ef} * {\dot{P}}_{e} ( t ) ;$$

$${\dot{P}}_{e} ( s ) = \frac{s}{( s + {p}_{1} ) ( s + {p}_{2} ) ( β s + 1 )} {P}_{e} ( s ) ;$$

$${Q}_{y} ( s ) = {T}_{1} ( s ) * {Q}_{v} ( s ) + {T}_{2} ( s ) * {P}_{y} ( s ) + {E}_{Qy} ( s ) ;$$

$${T}_{1} ( s ) = α \frac{s + {z}_{1}}{( s + {p}_{3} ) ( s + {p}_{4} )} ; and$$

$${T}_{2} ( s ) = - m * {T}_{1} ( s ) * \frac{s}{( s + {p}_{5} ) ( s + {p}_{6} )} .$$

Next, method 300 sends, saves, and/or displays these estimates, 308. In one embodiment, the estimates are sent to a display and listed upon the display. In an embodiment, the estimates are sent to a controller. The controller may utilize the estimates to control other ventilator components or to adjust the adaptive flow sensor model. In another embodiment, the estimates are sent from the memory to a display based on a inputted user command or pre-set command.

Method 300 includes a first determination operation 310 that determines if a command is still being received. Upon determination that a command is being received, method 300 repeats the running of the adaptive flow sensor model, 304. Upon determination that a command is not being received, method 300 ends, 312. The duration of the command may correspond with the time interval that the proximal flow sensor is offline. In alternative embodiment, duration of the command may correspond with the time interval that the proximal flow sensor has a reading of zero or another value designated to indicate sensor in “non-measuring” mode. In another embodiment, duration of the command may correspond with the time interval that the proximal flow is turned off. In a further embodiment, the duration of the command is a pre-set time interval entered by a user and/or programmed into the ventilator. In an additional embodiment, the duration of the command may correspond with the duration of time it takes the proximal flow sensor to recalibrate (auto-zeroing).

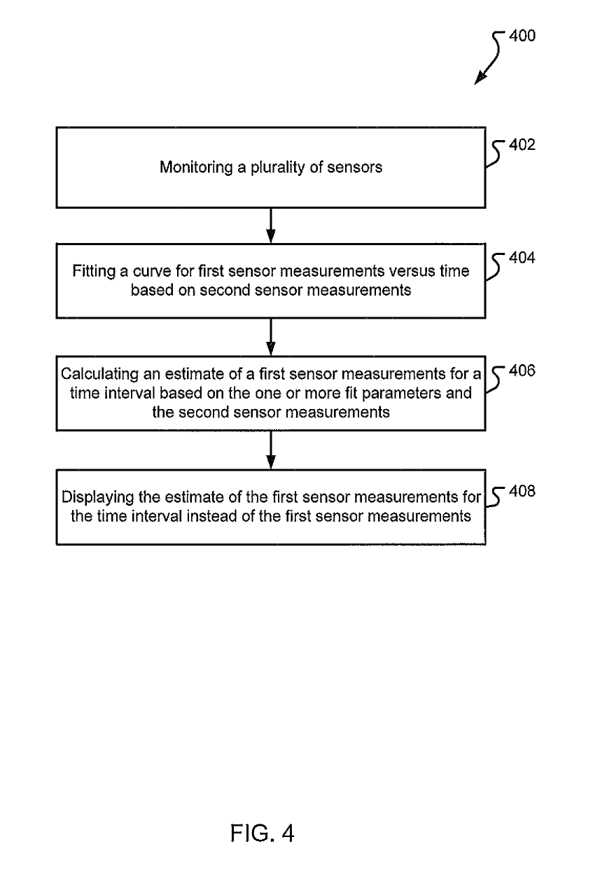

FIG. 4 represents an embodiment of a method for estimating at least one patient reading from at least one circuit sensor in a medical ventilator providing ventilation to a patient, 400.

As illustrated, method 400 monitors a plurality of sensors including a first sensor and at least one second sensor to obtain first sensor measurements and second sensor measurements, 402. In one embodiment, the first sensor is the proximal flow sensor. In an alternative embodiment, the first sensor is the pressure sensor. In an another embodiment, the second sensor is a pressure sensor. In an additional embodiment, the second sensor(s) measures the other inputs of the model and may be measured by one or more sensors of the ventilator.

Utilizing this information, method 400 fits a curve for first sensor measurements versus time based on the second sensor measurements, 404. The model for the curve may be any suitable model as long as it can provide a reasonably accurate prediction of the pressure and/or flow at the wye based on past proximal sensor reading and current and/or past sensor readings for other sensors. In one embodiment, the model equations for the fitted curve are:

$${P}_{y} ( t ) = {P}_{exh} ( t ) + {Q}_{c} ( t ) * ( {K}_{1} + {K}_{2} * {Q}_{c} ( t ) ) ;$$

$${Q}_{c} ( t ) = {Q}_{exh} ( t ) + {C}_{ef} * {\dot{P}}_{e} ( t ) ;$$

$${\dot{P}}_{e} ( s ) = \frac{s}{( s + {p}_{1} ) ( s + {p}_{2} ) ( β s + 1 )} {P}_{e} ( s ) ;$$

$${Q}_{y} ( s ) = {T}_{1} ( s ) * {Q}_{v} ( s ) + {T}_{2} ( s ) * {P}_{y} ( s ) + {E}_{Qy} ( s ) ;$$

$${T}_{1} ( s ) = α \frac{s + {z}_{1}}{( s + {p}_{3} ) ( s + {p}_{4} )} ; and$$

$${T}_{2} ( s ) = - m * {T}_{1} ( s ) * \frac{s}{( s + {p}_{5} ) ( s + {p}_{6} )} .$$

The fitting relies on one or more fit parameters. The values of said one or more fit parameters are found by said fitting. The fit parameters may be constants chosen based on the specific patient type, the ventilator application, and other ventilator parameters.

Method 400 calculates an estimate of a first sensor measurements for a time interval based on the one or more fit parameters and second sensor measurements, 406. In one embodiment, the time interval is the time that the proximal flow sensor is offline. In alternative embodiment, the time interval is the time that the proximal flow sensor has a reading of zero. In another embodiment, the time interval is the time that the proximal flow sensor is turned off. In a further embodiment, the time interval is pre-set time entered by a user and/or programmed into the ventilator. In an additional embodiment, the time interval is equal to the duration of time it takes the proximal flow sensor to recalibrate (auto-zeroing).

In an alternative embodiment, the time interval is the time that the pressure sensor is offline. In an embodiment, the time interval is the time that the pressure sensor has a reading of zero. In another embodiment, the time interval is the time that the pressure sensor is turned off.

The estimate of the first sensor measurements for the time interval is displayed by method 400 instead of the first sensor measurements, 408. The displaying step of method 400 may further include displaying the estimate of the first sensor measurements for the time interval instead of the first sensor measurements when the first sensor measurements are a predetermined value. In an alternative embodiment, the displaying step of method 400 includes displaying the first sensor measurements for the time interval only when the first sensor measurements are not the predetermined value. In one embodiment, the displaying step of method 400 includes displaying the estimate of the first sensor measurements for the time interval instead of the first sensor measurements when the ventilator is performing a predetermined action. In addition to the previous step, the displaying step of method 400 may also include detecting a purge of sensor lines associated with the first sensor and displaying the estimate of the first sensor measurements for the time interval instead of the first sensor measurements when the ventilator is performing a purge of sensor lines associated with the first sensor.

In another embodiment, the displaying step of Method 400 includes displaying the estimate of the first sensor measurements for the time interval instead of the first sensor measurements when the first sensor is turned off. In a further embodiment, the displaying step of Method 400 includes displaying the estimate of the first sensor measurements for the time interval instead of the first sensor measurements when the first sensor measurements are about zero.

### EXAMPLES

### Example 1

The following equations express the current discretized implementation of the NPB 840 ventilator for the neonatal patient setting. The variable “n” is equal to interval of measurement. In one embodiment, is used to count discrete intervals of 10 or 5 milliseconds (ms) each. The NPB 840 ventilator utilizes a 5 ms sampling interval and characterizes the components of the tubing including patient circuit resistance and compliance. In this implementation, E_{Qy} is assumed negligible.

P_{y}(n)=Pₑₓₕ(n)+Q_{c}(n)*(K₁+K₂*Q_{c}(n));

Q_{c}(n)=Qₑₓₕ(n)+C_{ef}*\dot{P}ₑ(n);

\dot{P}ₑ(n)=0.185*(P_{fe}(n)−P_{fe}(n−1))+0.0745*Pₑ(n−1)−0.000023*\dot{P}ₑ(n−2)

P_{fe}(n)=0.65*(P_{fe}(n−1)+0.35*Pₑ(n); P_{fe}(0)=0.0

\dot{P}_{y}(n)=0.043*((P_{y}(n)−P_{y}(n−1))+0.8714*\dot{P}_{y}(n−1)−0.0884*\dot{P}_{y}(n−2)

Q₁(n)=Qᵥ(n)−m*\dot{P}_{y}(n)

Q₂(n)=0.75*Q₂(n−1)+0.25*Q₁(n)

Q₃(n)=A1*Qᵥ(n−1)+A2*Q₂(n)−A3*Q₂(n−1)

$$A 1 = \frac{1}{1 + 0.005 * c}$$

$$A 2 = \frac{a * ( 1 + 0.005 * b )}{1 + 0.005 * c}$$

$$A 3 = \frac{a}{1 + 0.005 * c}$$

Model parameters a, b, c, and m are dynamically updated to optimize the match between actual and simulated readings over a regular cycle, such as one breath. Additionally, one or more of these parameters may assume different values depending on the breath phase (inhalation or exhalation). In this example for neonatal patients, b, c, and m were fixed as follows: b=2.0; c=2.5; and m=0.1. Inspiratory and expiratory values for “a” (aᵢₙₛₚ, aₑₓₚ) were determined in a two step process. First aᵢₙₛₚ and aₑₓₚ where determined by utilizing the following equations:

aᵢₙₛₚ=(peak inspiratory proximal flow, actual)/(peak inspiratory proximal flow, simulated); and

aₑₓₚ=(peak expiratory proximal flow, actual)/(peak expiratory proximal flow, simulated).

Second aᵢₙₛₚ, aₑₓₚ were fine tuned to minimize inspiratory (Vₜᵢ) and expiratory (Vₜₑ) volume errors between the actual and simulated results. Also, when aᵢₙₛₚ, aₑₓₚ assume different values, care should be taken to reset initial inspiratory or expiratory input values to ensure a smooth transition between parameter modeling in inhalation and exhalation phases. Steps 1 and 2 may be combined to optimize a weighted cost function, such as:

Determine “a” such that:

Minimize [ω₁*abs(PeakFlowDifference)+ω₂*abs(Volume Difference)]; abs[ ]=absolute value function.

ω₁ and ω₂ are weighing coefficients to assign relative priority. As previously discussed, multiple model parameters and more involved optimization strategies may be used as suitable for application needs. Furthermore, other wave-shaping modeling approaches and waveform quantification and modeling techniques may be utilized. Moreover, parameters of such models may be dynamically updated and optimized during normal sensor operation to obtain the best fit between the actual and simulated signals.

Numerous other changes may be made which will readily suggest themselves to those skilled in the art and which are encompassed in the spirit of the disclosure and as defined in the appended claims. While various embodiments have been described for purposes of this disclosure, various changes and modifications may be made which are well within the scope of the present invention. For example, if the capabilities of the system allow all sensor tubes to be purged and all missing sensor data to be estimated simultaneously, thus reducing the overall time necessary to complete the purge cycle at the expense of purge system cost. Numerous other changes may be made which will readily suggest themselves to those skilled in the art and which are encompassed in the spirit of the disclosure and as defined in the appended claims.

What is claimed is:

1. A method for estimating at least one patient reading from at least one circuit sensor in a medical ventilator providing ventilation to a patient, the method comprising:
monitoring a plurality of sensors including a first sensor and at least one second sensor to obtain first sensor measurements and second sensor measurements;
fitting a curve for first sensor measurements versus time based on the second sensor measurements, wherein said fitting relies on one or more fit parameters, and wherein the values of said one or more fit parameters are found by said fitting;
calculating an estimate of first sensor measurements for a time interval based on the one or more fit parameters and the second sensor measurements; and
displaying the estimate of the first sensor measurements for the time interval instead of the first sensor measurements.

2. The method of claim 1 wherein displaying further comprising:
displaying the estimate of the first sensor measurements for the time interval instead of the first sensor measurements when the first sensor measurements are a predetermined value.

3. The method of claim 1 further comprising:
displaying the first sensor measurements for the time interval only when the first sensor measurements are not the predetermined value.

4. The method of claim 1 wherein displaying further comprising:
displaying the estimate of the first sensor measurements for the time interval instead of the first sensor measurements when the ventilator is performing a predetermined action.

5. The method of claim 3 wherein displaying further comprising:
detecting a purge of sensor lines associated with the first sensor; and
displaying the estimate of the first sensor measurements for the time interval instead of the first sensor measurements when the ventilator is performing a purge of sensor lines associated with the first sensor.

6. The method of claim 1 wherein displaying further comprising:
displaying the estimate of the first sensor measurements for the time interval instead of the first sensor measurements when the first sensor is turned off.

7. The method of claim 1 wherein displaying further comprising:
displaying the estimate of the first sensor measurements for the time interval instead of the first sensor measurements when the first sensor measurements are about zero.

8. The method of claim 1 wherein model equations (in time and frequency domains) for the curve fitted are:
$${P}_{y} ( t ) = {P}_{exh} ( t ) + {Q}_{c} ( t ) * ( {K}_{1} + {K}_{2} * {Q}_{c} ( t ) ) ;$$
$${Q}_{c} ( t ) = {Q}_{exh} ( t ) + {C}_{ef} * {\dot{P}}_{e} ( t ) ;$$
$${\dot{P}}_{e} ( s ) = \frac{s}{( s + {p}_{1} ) ( s + {p}_{2} ) ( β s + 1 )} {P}_{e} ( s ) ;$$
$${Q}_{y} ( s ) = {T}_{1} ( s ) * {Q}_{v} ( s ) + {T}_{2} ( s ) * {P}_{y} ( s ) + {E}_{Qy} ( s ) ;$$
$${T}_{1} ( s ) = α \frac{s + {z}_{1}}{( s + {p}_{3} ) ( s + {p}_{4} )} ; and$$
$${T}_{2} ( s ) = - m * {T}_{1} ( s ) * \frac{s}{( s + {p}_{5} ) ( s + {p}_{6} )} .$$

9. A pressure support system comprising:
a processor;
a pressure generating system adapted to generate a flow of breathing gas controlled the processor;
a ventilation system including a patient circuit controlled by the processor;
at least one circuit sensor in fluid controlled by the patient circuit via one or more sensor tubes; and
an adaptive flow sensor model controlled by the processor, the adaptive flow sensor model is adapted to estimate patient sensor data during situations in which the at least one circuit sensor cannot obtain a reading.

10. The pressure support system of claim 9, wherein the adaptive flow sensor model is controlled by the circuit sensor.

11. The pressure support system of claim 9, wherein the adaptive flow sensor model is controlled by the ventilation system.

12. The pressure support system of claim 9, further comprising an autozeroing mechanism controlled by the processor and adapted to recalibrate the at least one sensor circuit discontinuing any readings of the least one circuit sensor; and

13. The pressure support system of claim 9, further comprising a sensor tube purge model in controlled by the processor and adapted to initiate a purge cycle that purges the sensor tubes, wherein each purge cycle includes repeatedly discharging gas through the sensor tubes into the patient circuit and discontinuing any readings of the least one circuit sensor.

14. The pressure support system of claim 9, wherein the adaptive flow sensor model is adapted to utilize the following model equations to estimate patient sensor data:
$${P}_{y} ( t ) = {P}_{exh} ( t ) + {Q}_{c} ( t ) * ( {K}_{1} + {K}_{2} * {Q}_{c} ( t ) ) ;$$
$${Q}_{c} ( t ) = {Q}_{exh} ( t ) + {C}_{ef} * {\dot{P}}_{e} ( t ) ;$$
$${\dot{P}}_{e} ( s ) = \frac{s}{( s + {p}_{1} ) ( s + {p}_{2} ) ( β s + 1 )} {P}_{e} ( s ) ;$$
$${Q}_{y} ( s ) = {T}_{1} ( s ) * {Q}_{v} ( s ) + {T}_{2} ( s ) * {P}_{y} ( s ) + {E}_{Qy} ( s ) ;$$
$${T}_{1} ( s ) = α \frac{s + {z}_{1}}{( s + {p}_{3} ) ( s + {p}_{4} )} ; and$$
$${T}_{2} ( s ) = - m * {T}_{1} ( s ) * \frac{s}{( s + {p}_{5} ) ( s + {p}_{6} )} .$$

15. The pressure support system of claim 9, further comprising a display controlled by the processor, the display is adapted to display the estimated patient sensor data.

16. A medical ventilator system, comprising:
a processor;
a gas regulator controlled by the processor, the gas regulator adapted to regulate a flow of gas from a gas supply to a patient via a patient circuit;
a flow sensor package disposed in the patient circuit, the flow sensor package controlled by a gas accumulator;
a pressure sensor coupled to the gas accumulator and controlled by the processor, the pressure sensor adapted to provide pressure readings in the gas accumulator to the processor; and
an adaptive flow sensor model controlled by the processor for estimating patient sensor data when the flow sensor and pressure sensor are not reading patient data.

17. The medical ventilator system of claim 16, wherein the adaptive flow sensor model is controlled by the circuit sensor.

18. The medical ventilator system of claim 16, wherein the adaptive flow sensor model is controlled by the ventilation system.

19. The medical ventilator system of claim 16, wherein the adaptive flow sensor model is adapted to utilize the following model equations to estimate patient sensor data:
$${P}_{y} ( t ) = {P}_{exh} ( t ) + {Q}_{c} ( t ) * ( {K}_{1} + {K}_{2} * {Q}_{c} ( t ) ) ;$$
$${Q}_{c} ( t ) = {Q}_{exh} ( t ) + {C}_{ef} * {\dot{P}}_{e} ( t ) ;$$
$${\dot{P}}_{e} ( s ) = \frac{s}{( s + {p}_{1} ) ( s + {p}_{2} ) ( β s + 1 )} {P}_{e} ( s ) ;$$
$${Q}_{y} ( s ) = {T}_{1} ( s ) * {Q}_{v} ( s ) + {T}_{2} ( s ) * {P}_{y} ( s ) + {E}_{Qy} ( s ) ;$$
$${T}_{1} ( s ) = α \frac{s + {z}_{1}}{( s + {p}_{3} ) ( s + {p}_{4} )} ; and$$
$${T}_{2} ( s ) = - m * {T}_{1} ( s ) * \frac{s}{( s + {p}_{5} ) ( s + {p}_{6} )} .$$

20. The medical ventilator system of claim 16, further comprising a display controlled by the processor, the display is adapted to display the estimated patient sensor data.
